(19) United States
(12) Patent Application Publication
(10) Pub. No.: US 2026/0248995 A1
(43) Pub. Date: Aug. 27, 2026

(54) BILE DRAINAGE AND REINFUSION DEVICE

(71) Applicant: NANJING DRUM TOWER HOSPITAL, Nanjing (CN)

(72) Inventors: Yanping CAO, Nanjing (CN); Qian WANG, Nanjing (CN); Hua YANG, Nanjing (CN); Juan TIAN, Nanjing (CN); Xiu WEN, Nanjing (CN)

(73) Assignee: NANJING DRUM TOWER HOSPITAL, Nanjing (CN)

(21) Appl. No.: 19/575,985

(22) Filed: Mar. 24, 2026

Related U.S. Application Data
(63) Continuation of application No. PCT/CN2025/129442, filed on Oct. 23, 2025.

(30) Foreign Application Priority Data
Feb. 27, 2025 (CN) 202510224873.9

(51) Int. Cl. A61M 1/00; B01D 29/13; B01D 35/30

(52) U.S. Cl. CPC A61M 1/77; A61M 1/60; A61M 1/79; A61M 1/84; B01D 29/13; B01D 35/306; A61M 2202/0403; A61M 2210/0618; B01D 2201/0453; B01D 2201/0461

(57) ABSTRACT

A bile drainage and reinfusion device includes a nasobiliary tube, a drainage bag, a sealed cylinder, a heating mechanism, a filtering mechanism, and a reinfusion tube, wherein the drainage bag is disposed at an output end of the nasobiliary tube, the sealed cylinder is connected to an output end of the drainage bag, the heating mechanism and the filtering mechanism are disposed inside the sealed cylinder to heat and filter drained bile, and the reinfusion tube is connected to an output end of the sealed cylinder to reinfuse the heated and filtered bile into a patient. The drained and reinfused bile is heated and filtered by a heating mechanism and a filtering mechanism, so as to remove and intercept impurities such as tissue fragments in bile, prevent the impurities from entering the patient, and prevent tubing blockage caused by bile viscosity.

### CROSS REFERENCE TO THE RELATED APPLICATIONS

This application is the continuation application of International Application No. PCT/CN2025/129442, filed on October 23, 2025, which is based upon and claims priority to Chinese Patent Application No. 202510224873.9, filed on February 27, 2025, the entire contents of which are incorporated herein by reference.

### TECHNICAL FIELD

The present disclosure belongs to the technical field of bile drainage and reinfusion, and specifically relates to a bile drainage and reinfusion device.

### BACKGROUND

With the in-depth study of the biological behaviors of severe acute pancreatitis and gastric cancer, it has been found that early autologous bile (gastric juice) reinfusion is conducive to the recovery of gastrointestinal function and structure, helps maintain the integrity of the intestinal mucosa and protects the intestinal mucosal barrier, can significantly reduce the occurrence of enterogenic infection, and also exerts a significant improving effect on the functional recovery of intra-abdominal organs after abdominal surgery.

In the prior art, for patients with long-term nasobiliary drainage tube placed in biliary tract tumors, the daily volume of drained bile is considerable, and particularly high bile drainage output results in a massive loss of body fluids and water. Patients with long-term external bile drainage are prone to loss of appetite, diarrhea, water-electrolyte and acid-base imbalance, insufficient circulating blood volume, etc. Such conditions affect the digestion of fats and the absorption of fat-soluble vitamins, impair postoperative recovery, and thus prolong hospital stay and increase medical costs. However, reinfusion of bile into the duodenum can avoid massive loss of body fluids, electrolytes, digestive juices and other substances, correct electrolyte imbalance, improve appetite, and accelerate the postoperative rehabilitation of patients.

According to the bile reinfusion devices in the prior art, first, the drained bile needs to be collected in a container from a drainage bag, then the bile is filtered with sterile gauze, and then the filtered bile is collected in another container and connected to an infusion set. the bile is input via a nasointestinal tube or jejunostomy tube. During operation, bile may be contaminated by bacteria and fails to be maintained at a constant temperature. In addition, medical personnel are exposed to a high risk of contact with the body fluids of the patients, which constitutes technical disadvantages unfavorable to their self-protection.

Chinese Patent No. 201710907318 .1 discloses a disposable closed bile drainage and reinfusion device, including a drainage tube 1, an umbrella shaped murphy drip chamber 2, a collection bag 3, and a transition tube 5, where a front end of the umbrella shaped murphy drip chamber 2 is connected to the drainage tube 1, a rear end of the umbrella shaped murphy drip chamber 2 is connected to one end of the transition tube 5, and the other end of the transition tube 5 is connected to the collection bag 3. The above components integrally form the closed bile drainage and reinfusion device. This bile drainage and reinfusion device renders the bile collection and reinfusion process a closed operation, which minimizes iatrogenic contamination of the bile and contamination of the surrounding environment and operators by the bile, thereby providing dual protection for both patients and medical personnel. However, this technical solution lacks filtration of bile. Moreover, the bile is notat a constant temperature, such that this solution needs to be improved.

### SUMMARY

An objective of the present disclosure is to provide a bile drainage and reinfusion device, which solves the technical problem that in the prior art, the bile drainage and reinfusion devices lack a structure that filters, heats and insulates the bile.

To fulfill the purpose of the above invention, the invention provides the following technical solution:

Firstly, the present disclosure provides a bile drainage and reinfusion device, including a nasobiliary tube, a drainage bag, a sealed cylinder, a heating mechanism, a filtering mechanism, and a reinfusion tube, wherein the drainage bag is disposed at an output end of the nasobiliary tube, the sealed cylinder is connected to an output end of the drainage bag, the heating mechanism and the filtering mechanism are disposed inside the sealed cylinder to heat and filter drained bile, and the reinfusion tube is connected to an output end of the sealed cylinder to reinfuse the heated and filtered bile into a patient.

The heating mechanism and the filtering mechanism are disposed to heat and filter the drained and reinfused bile to remove and intercept impurities such as tissue fragments in bile, prevent the impurities from entering the patients, and prevent tubing blockage caused by bile viscosity. By heating the bile, discomfort caused by low-temperature bile entering the human body is reduced, and the activity of enzymes in the bile is maintained. Moreover, the filtered and heated bile is delivered through the reinfusion tube, thus forming an integrated closed drainage and reinfusion device to minimize iatrogenic contamination of bile and prevent contamination of the surrounding environment and operators by the bile.

Preferably, the heating mechanism includes a heating layer and a plurality of electric heating rods, the heating layer is cylindrically disposed inside the sealed cylinder, the plurality of electric heating rods are disposed in a circumferential array inside the heating layer, and the heating layer is filled with water for heating. By disposing the heating layer, water is heated via the electric heating rods inside the heating layer, such that the bile entering therein is heated. The uniformity of the bile heating temperature is improved by way of water bath heating, the water temperature is easily controlled, and the control precision is higher. Moreover, water bath heating of the bile is relatively mild and prevents changes in the properties of the bile caused by direct heating.

Preferably, the filtering mechanism includes a first disc, a second disc, and a plurality of filter tubes, the first disc and the second disc are spaced apart inside an input end of the sealed cylinder, the plurality of filter tubes are disposed in a circumferential array on the first disc and the second disc, and the filter tubes on the first disc and the second disc filter the bile entering the first disc and the second disc. By disposing the first disc and the second disc at the input end of the sealed cylinder, and the filter tubes disposed on the first disc and the second disc filters the bile therein and removes and eliminates impurities in the bile.

Preferably, the filter tubes include coarse filter tubes and fine filter tubes, the coarse filter tubes and fine filter tubes are both disposed in a circumferential array on the first disc and the second disc, the coarse filter tubes on the first disc are in communication with tube ports of the fine filter tubes on the second disc, and the fine filter tubes on the first disc are in communication with tube ports of the coarse filter tubes on the second disc. By disposing the coarse filter tubes and the fine filter tubes disposed previously in a staggered and spaced manner relative to each other, the coarse filter tubes and the fine filter tube disposed on the first disc and the second disc are oppositely disposed, and the filter tubes are dredged by pushing the first disc and the second disc.

Preferably, the filtering mechanism further includes a spring coil and two side surfaces of the spring coil are fixedly disposed on the first disc and the second disc. The first disc and the second disc are connected by disposing the spring coil, and are limited when being pressed for dredging.

Secondly, the present disclosure also provides a bile drainage and reinfusion device, including a nasobiliary tube, a drainage bag, a heating mechanism, a filtering mechanism, a reinfusion tube, and a circulation tube, wherein the drainage bag is disposed at an output end of the nasobiliary tube, the filtering mechanism is connected to an output end of the drainage bag, the heating mechanism is connected to an output end of the filtering mechanism and is configured to heat filtered bile, the reinfusion tube is disposed at an output end of the heating mechanism, the circulation tube is disposed at a tail end of the filtering mechanism, and an output end of the circulation tube is in communication with an input end of the filtering mechanism.

By disposing the heating mechanism and the filtering mechanism, the bile is filtered and heated successively. By disposing the circulation tube, whether the bile is circularly filtered is determined to obtain purer bile.

Preferably, the filtering mechanism includes a mounting cylinder, a mounting base, a plurality of ceramic filter tubes, and a sealing cap, the mounting base is in communication with the output end of the drainage bag, the mounting cylinder is hermetically disposed on the mounting base, the plurality of ceramic filter tubes are disposed in a circumferential array on the mounting base, and the sealing cap is hermetically disposed on the plurality of ceramic filter tubes. By using the ceramic filter tubes, bile filtration is accelerated by means of the cross-flow filtration principle, while the risk of clogging the filter tube is reduced.

Preferably, the filtering mechanism further includes a nut and a bearing, the bearing is sleeved on an outer periphery of the sealing cap, the nut is disposed on an outer periphery of the bearing, an end of the mounting cylinder is provided with a thread adapted to the nut, and the nut drives the sealing cap via the thread to be fixed on the mounting cylinder. By disposing the bearing, the nut is fixed on the sealing cap via the thread.

Preferably, the heating mechanism includes a plurality of electric heating rods, an outer cylinder, and an inner cylinder, the outer cylinder is connected to an output end of the filtering mechanism, the inner cylinder is disposed inside the outer cylinder, and the plurality of electric heating rods are disposed in a circumferential array in a gap composed of the outer cylinder and the inner cylinder. By disposing the electric heating rods in the gap between the outer cylinder and the inner cylinder, the bile is heated by way of water bath heating.

Preferably, the heating mechanism further includes a support base, a rotating barrel, and a plurality of arc-shaped plates, the support base is disposed inside the inner cylinder, the rotating barrel is movably sleeved on an outer periphery of the support base, and the plurality of arc-shaped plates are disposed in a circumferential array on an outer periphery of the rotating barrel.

Compared with the prior art, the present disclosure has the following beneficial effects:

1. According to the bile drainage and reinfusion device provided by the present disclosure, the heating mechanism and the filtering mechanism are disposed to heat and filter the drained and reinfused bile to remove and intercept impurities such as tissue fragments in bile, prevent the impurities from entering the patient, and prevent tubing blockage caused by bile viscosity. By heating the bile, discomfort caused by low-temperature bile entering the human body is reduced, and the activity of enzymes in the bile is maintained.

2. According to the bile drainage and reinfusion device provided by the present disclosure, by disposing the heating layer, water is heated via the electric heating rods inside the heating layer, such that the bile entering therein is heated. The uniformity of the bile heating temperature is improved by way of water bath heating, the water temperature is easily controlled, and the control precision is higher. Moreover, water bath heating of the bile is relatively mild and prevents changes in the properties of the bile caused by direct heating.

3. According to the bile drainage and reinfusion device provided by the present disclosure, filter tubes disposed on the first disc and the second disc filters the bile therein and removes and eliminates impurities in the bile. By disposing the coarse filter tubes and the fine filter tubes disposed previously in a staggered and spaced manner relative to each other, the coarse filter tubes and the fine filter tube disposed on the first disc and the second disc are oppositely disposed, and the filter tubes are dredged by pushing the first disc and the second disc.

4. According to the bile drainage and reinfusion device provided by the present disclosure, by disposing the circulation tube, whether the bile is circularly filtered is determined to obtain purer bile, and by using the ceramic filter tube, bile filtration is accelerated by means of the cross-flow filtration principle, while the risk of clogging the filter tube is reduced.

### BRIEF DESCRIPTION OF THE DRAWINGS

FIG. 1 is a schematic structural diagram in Embodiment I of the present disclosure.

FIG. 2 is an enlarged schematic diagram of a heating mechanism in Embodiment I of the present disclosure.

FIG. 3 is an enlarged structural schematic diagram of a filtering mechanism in Embodiment I of the present disclosure.

FIG. 4 is a schematic structural diagram in Embodiment II of the present disclosure.

FIG. 5 is an enlarged schematic diagram of a heating mechanism in Embodiment II of the present disclosure.

FIG. 6 is an exploded schematic structural diagram of the heating mechanism in Embodiment II of the present disclosure.

FIG. 7 is an exploded schematic structural diagram of a filtering mechanism in Embodiment II of the present disclosure.

In the figure: 1-Nasobiliary tube, 2-Drainage bag, 3-Sealed cylinder, 4-Heating mechanism, 41-Heating layer, 42-Electric heating rods, 43-Outer cylinder, 44-Inner cylinder, 45-Support base, 46-Rotating barrel, 47-Arc-shaped plates, 5-Filtering mechanism, 51-Mounting cylinder, 52-Mounting base, 53-Threaded holes, 54-Ceramic filter tubes, 55-Sealing cap, 56-Nut, 57-Bearing, 511-First disc, 512-Second disc, 513-Spring coil, 514-Coarse filter tubes, 515-Fine filter tubes, 6-Reinfusion tube, 7-Speed regulating valve, 8-Circulation tube, 9-Circulation pump, 10-First control valve, 11-Flushing connector, 12-First filtrate tube, 13-Second filtrate tube, 14-Confluence tube, 15-Second control valve.

### DETAILED DESCRIPTION OF THE EMBODIMENTS

The present disclosure will be further described below in conjunction with drawings. The following embodiments are merely intended to illustrate the technical solutions of the present disclosure more clearly, and shall not be used to limit the protection scope of the present disclosure.

### Embodiment I

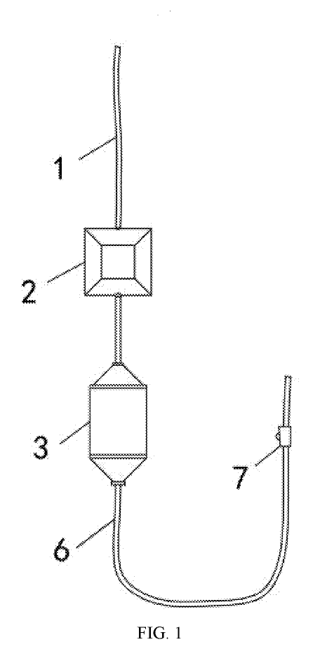

As shown in FIGS. 1-3, provided is a bile drainage and reinfusion device, including a nasobiliary tube 1, a drainage bag 2, a sealed cylinder 3, and a reinfusion tube 6, where the nasobiliary tube 1 is preferably a product in the prior art, an input end of the drainage bag 2 is connected to the nasobiliary tube 1, an output end of the drainage bag 2 is connected to an input end of the sealed cylinder 3 via a hose in the prior art, and the reinfusion tube 6 is hermetically connected to an output end of the sealed cylinder 3.

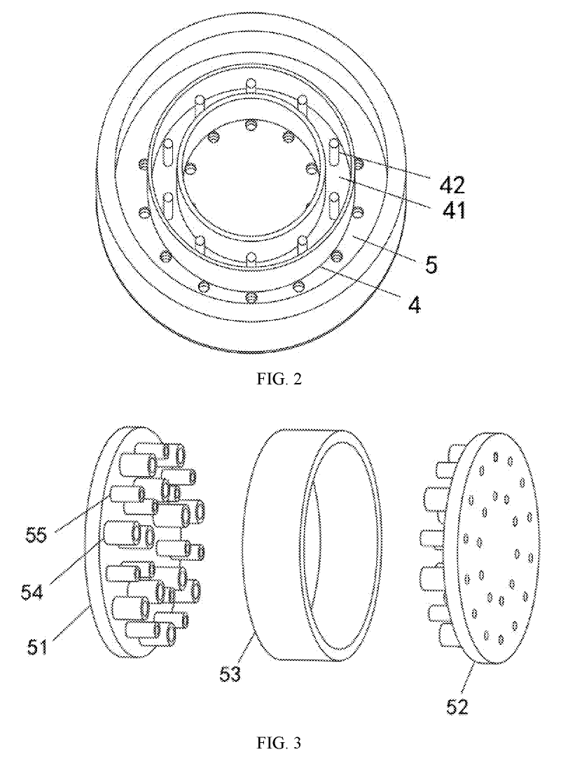

As shown in FIGS. 1 and FIG. 2, the sealed cylinder 3 in this embodiment includes a heating mechanism 4 and a filtering mechanism 5, where the filtering mechanism 5 is disposed at a position inside the sealed cylinder 3 close to the input end, the heating mechanism 4 is disposed inside the sealed cylinder 3, and during use, the bile in the drainage bag 2 filtered and heated by the filtering mechanism 5 and the heating mechanism 4 in the sealed cylinder 3 successively is reinfused into the patient via the reinfusion tube 6. Impurities entering the patient are reduced by means of filtration, and by heating the bile, discomfort caused by low-temperature bile entering the human body is reduced, and the activity of enzymes in the bile is maintained.

As shown in FIGS. 1, FIG. 2, and FIG. 3, the filtering mechanism 5 in this embodiment includes a first disc 511 and a second disc 512, where the first disc 511 is fixed at a position inside the sealed cylinder 3 close to the input end, filter tubes are fixed on both the first disc 511 and the second disc 512, the filter tubes on the first disc 511 and the second disc 512 are communicated and aligned, and during use, the bile entering the sealed cylinder 3 enters into the holes in the second disc 512 from the holes in the first disc 511 via the filter tubes and enters the heating mechanism 4.

As shown in FIG. 3, the filter tubes in this embodiment are preferably coarse filter tubes 514 and fine filter tubes 515 with different diameters. The inner diameter of the coarse filter tubes 514 is the outer diameter of the fine filter tubes 515. The plurality of coarse filter tubes 514 and fine filter tubes 515 are fixed in a circumferential array on opposite surfaces of the first disc 511 and the second disc 512. The coarse filter tubes 514 and fine filter tubes 515 are fixed in a staggered manner relative to each other, i.e., one fine filter tube 515 or coarse filter tube 514 is fixed between two coarse filter tubes 514 or fine filter tubes 514. The spring coil 513 is connected between the first disc 511 and the second disc 512 in this embodiment. The spring coil 513 is preferably a helical spring plate structure in the prior art.

During use, the bile entering the sealed cylinder 3 first enters the coarse filter tubes 514 or fine filter tubes 515 via the holes in the first disc 511, and then enters the holes in the second disc 512 via the fine filter tubes 515 or coarse filter tubes 514 on the second disc 512 and then enters the heating mechanism 4. In this embodiment. When the coarse filter tubes 514 are blocked, after the device is detached, by pressing the first disc 511 and the second disc 512, the fine filter tubes 515 fixed thereon enter the inner holes in the coarse filter tubes 514 to be aligned for dredging. Pressing is limited by the disposed spring coil 513, so as to prevent damage due to overpressure.

As shown in FIGS. 1 and FIG. 2, the heating mechanism 4 in this embodiment includes a heating layer 41, where the heating layer 41 is preferably of a hollow cavity structure and is annularly fixed inside the sealed cylinder 3, with the second disc 512 fitting the end. The holes of the filter tubes on the second disc 512 are respectively located on both sides of the heating layer 41. In this embodiment, the heating layer 41 is filled with water, a plurality of electric heating rods 42 are disposed in a circumferential array inside the heating layer 41, and the electric heating rods 42 are preferably electric heating rods that are directly applied in the prior art.

During use, the bile filtered enters the sealed cylinder 3 via the filter tubes on the second disc 512. Water located in the heating layer 41 is preheated to a preset temperature under the action of the electric heating rods 42. The bile entering therein is heated by water bath heating, the uniformity of the bile heating temperature is improved, the water temperature is easily controlled, and the control precision is higher. Moreover, water bath heating of the bile is relatively mild and prevents changes in the properties of the bile caused by direct heating.

It should be noted that in this embodiment, to achieve an adjustable flow rate during bile reinfusion, a speed regulating valve 7 is additionally disposed on the reinfusion tube 6. The speed regulating valve 7 is preferably an infusion flow rate regulator that is directly applied in the prior art. During use, the flow rate during bile reinfusion is adjusted by sliding the roller disposed on the infusion flow rate regulator.

### Embodiment II

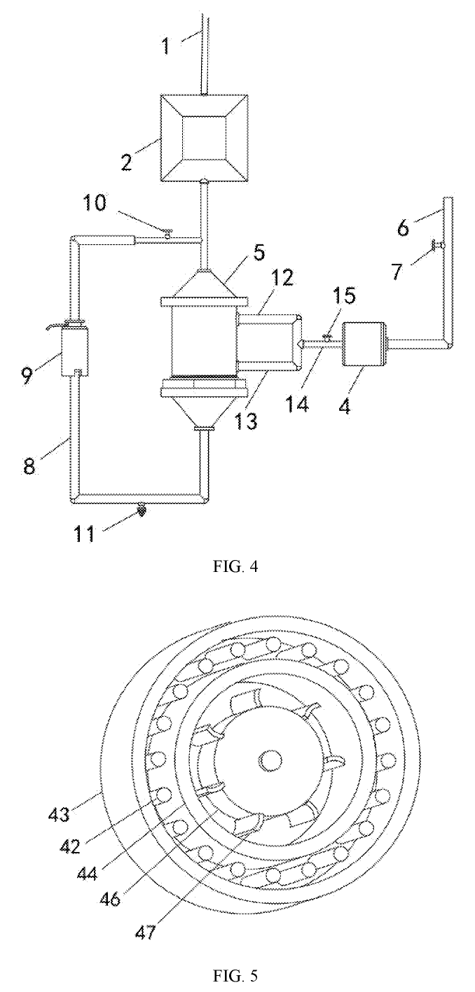

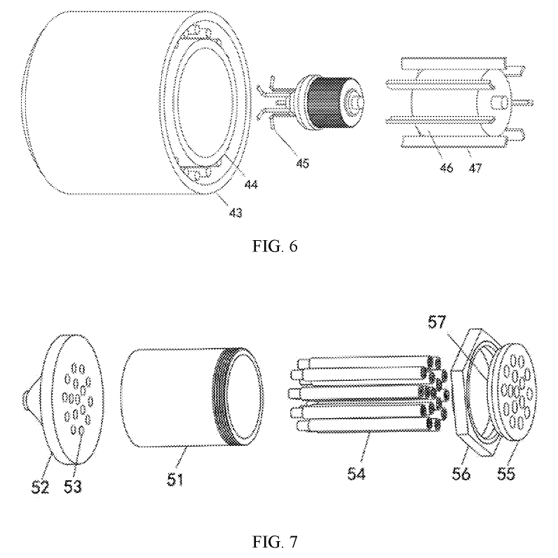

As shown in FIGS. 4-7, provided is a bile drainage and reinfusion device, including a nasobiliary tube 1, a drainage bag 2, a heating mechanism 4, a filtering mechanism 5, a reinfusion tube 6, and a circulation tube 8, where the nasobiliary tube 1 and the drainage bag 2 are preferably directly applied in the prior art. The drainage bag 2 is fixed and connected to an output end of the nasobiliary tube 1, the output end of the drainage bag 2 is preferably connected to the filtering mechanism 5 via a hose in the prior art, and the filtering mechanism 5 is preferably connected to the heating mechanism 4 via a hose in the prior art. The reinfusion tube 6 is connected and fixed at an output end of the heating mechanism 4. In this embodiment, a speed regulating valve 7 is preferably disposed on the reinfusion tube 6. The speed regulating valve 7 has the same effect with a structure in this embodiment, which is not repeatedly described herein. In this embodiment, the circulation tube 8 is disposed at a tail end of the filtering mechanism 5, and an output end of the circulation tube 8 is in communication with an input end of the filtering mechanism 5.

During use, the bile enters the filtering mechanism 5 successively via the nasobiliary tube 1 and the drainage bag 2. After the bile is filtered, the bile is repeatedly filtered and enters the circulation tube 8 until the bile is filtered thoroughly and enters the heating mechanism 4 for heating, or the primarily filtered bile is directly introduced into the heating mechanism 4 for heating, and then enters the reinfusion tube 6 to be reinfused into the patient. The bile is circularly filtered via the additionally disposed circulation tube 8, such that the purity of the reinfused bile is improved, and the impact of impurities is reduced.

As shown in FIG. 4, in this embodiment, the bile drainage and reinfusion device further includes a first filtrate tube 12 and a second filtrate tube 13, where the first filtrate tube 12 is preferably in communication with a position of the filtering mechanism 5 close to the top, the second filtrate tube 13 is preferably in communication with a position of the filtering mechanism 5 close to the bottom. Output ends of the first filtrate tube 12 and the second filtrate tube 13 are in communication with a confluence tube 14, and an output end of the confluence tube 14 is connected and fixed at the input end of the heating mechanism 4.

During use, in this embodiment, by disposing the upper and lower filtrate tubes, the filtered bile is transferred in double channels, which reduces the risk of blockage due to a single channel and improving the filtration rate of double channels.

Moreover, in this embodiment, the circulation tube 8 is further connected to a circulation pump 9 in the prior art. The bile in the filtering mechanism is circularly filtered via the disposed circulation pump 9. In this embodiment, a flushing connector 11 and a first control valve 10 are additionally disposed on the circulation tube 8, and a second control valve 15 is additionally disposed on the confluence tube 14.

During use, by opening the second control valve 15, closing the first control valve 10, and not starting the circulation pump 9, the filtered filtrate of the bile passing through the filtering mechanism 5 is input to the heating mechanism 4 successively via the first filtrate tube 12 and the second filtrate tube 13. The bile is filtered via primary filtration conveniently and rapidly.

Another filtration method is as follows: the second control valve 15 is opened, the first control valve 10 is opened, the circulation pump 9 is started, the filtrate of the primarily filtered bile passing through the filtering mechanism is input to the heating mechanism 4 for heating via the first filtrate tube 12 and the second filtrate tube 13, and moreover, the circulation pump 9 circularly filters the bile in the filtering mechanism 5, which increases filtration of the bile.

As shown in FIGS. 1 and FIG. 7, in this embodiment, the filtering mechanism 5 includes a mounting cylinder 51 and a mounting base 52. In this embodiment, the mounting cylinder 51 is sleeved and fixed on the mounting base 52 preferably by a welding technique in the prior art. The mounting base 52 is preferably of a conical structure in the prior art, where the top thereof is connected to the drainage bag 2 via a hose in the prior art, and the bottom thereof is provided with a plurality of threaded holes 53 in a circumferential array. A ceramic filter tube 54 is threadedly connected to each of the threaded holes 53. In this embodiment, the ceramic filter tube 54 is preferably a ceramic filter tube 54 in the prior art, and the pore diameter of the selected ceramic filter tube 54 is preferably 0.1-10 um.

In this embodiment, the ceramic filter tube 54 encircled by the mounting cylinder 51, where an outer periphery of an end of the mounting cylinder 51 is provided with a threaded groove, the end of the mounting cylinder 51 is provided with a sealing cap 55, the sealing cap 55 is provided with a plurality of through holes adapted to the ceramic filter tube 54, each of the through holes abuts against the end of the ceramic filter tube 54, and the bile passing through the ceramic filter tube 54 may be output via the through hole. In this embodiment, the outer periphery of the sealing cap 55 in the prior art is sleeved and fixed with the bearing 57 in the prior art, where a nut 56 is fixed to an outer ring of the bearing 57. In this embodiment, the diameter of the nut 56 is adapted to the outer diameter of the mounting cylinder 51, and the nut 56 is adapted to the threaded groove in the mounting cylinder 51.

During use, by threadedly fixing the ceramic filter tube 54 via the threaded hole 53 in the mounting base 52, the sealing cap 55 abuts against and is sleeved on the ceramic filter tube 54. Then the nut 56 is rotated. Under the action of the bearing 57, the nut 56 rotates on the threaded groove in the mounting cylinder 51 to drive the sealing cap 55 to abut against the ceramic filter tube 54.

As shown in FIGS. 4, FIG. 5, and FIG. 6, in this embodiment, the heating mechanism 4 includes an outer cylinder 43 and an inner cylinder 44, where the inner cylinder 44 is fixed in the outer cylinder 43 by a welding technique in the prior art. The confluence tube 14 penetrates into the inner cylinder 44. In this embodiment, a plurality of electric heating rods 42 are fixed in a circumferential array in a cavity encircled by the inner cylinder 44 and the outer cylinder 43. Moreover, the formed cavity is filled with water. Water bath heating is realized by way of heating by the electric heating rods 42. The structure and principle of the electric heating rods 42 are the same as those of a structure in Embodiment 1, which are not repeatedly described herein.

In this embodiment, a support base 45 is fixed inside the inner cylinder 44, where the support base 45 includes a support feet disposed in a circumferential array. A welding disc is fixed to the tops of the support feet. In this embodiment, a motor in the prior art is fixed to the top of the disc. A rotating barrel 46 is fixed to an output end of the motor. A plurality of arc-shaped plates 47 are fixed in a circumferential array on an outer periphery of the rotating barrel 46. In this embodiment, the rotating barrel 46 is preferably of a hollow cylindrical structure. The motor and the disc are sleeved inside, and the inner diameter of the rotating barrel 46 is adapted to the outer diameter of the disc. Moreover, to improve the sealing performance between the rotating barrel 46 and the disc, a sealing ring is fixed to the outer periphery of the disc, where the sealing ring is preferably a rubber ring in the prior art, and the rubber ring is hermetically connected to an inner wall of an end of the rotating barrel 46.

During use, the bile filtered by the ceramic filter tube 54 enters the inner cylinder 44 via the confluence tube 14. Water in the outer cylinder 43 has been heated to a preset temperature via the electric heating rods 42 before the bile enters the outer cylinder. The temperature is preferably 37-38℃. In this case, the motor is started to drive the rotating barrel 46 and the arc-shaped plates 47 thereon to rotate, so as to uniformly stir the bile in the inner cylinder 44 via the arc-shaped plates 47, thereby achieving heating uniformity and increasing the heating rate. Moreover, the bile is prevented from entering the rotating barrel 46 via the disposed sealing ring.

The operating principle is as follows: the bile enters the filtering mechanism 5 successively via the nasobiliary tube 1 and the drainage bag 2, the ceramic filter tube 54 therein filters the bile to remove and intercept impurities such as tissue debris in the bile, prevent the impurities from entering the patient, and prevent tubing blockage caused by bile viscosity. The bile filtered by the ceramic filter tube 54 enters the inner cylinder 44 via the confluence tube 14, where water in the outer cylinder 43 has been heated to a preset temperature via the electric heating rods 42 before the bile enters the outer cylinder. In this case, the motor is started to drive the rotating barrel 46 and the arc-shaped plates 47 thereon to rotate, so as to uniformly stir the bile in the inner cylinder 44 via the arc-shaped plates 47, thereby achieving heating uniformity. Then, the bile is reinfused to the patient via the reinfusion tube 6.

The embodiments of the present disclosure have been described above with reference to the accompanying drawings. However, the present disclosure is not limited to the aforesaid specific embodiments, which are merely illustrative rather than restrictive. A person of ordinary skill in the art may make numerous forms thereof under the enlightenment of the present disclosure without departing from the spirit of the present disclosure and the scope protected by the claims, and all such forms shall fall within the protection scope of the present disclosure.

What is claimed is:

1. A bile drainage and reinfusion device, comprising a nasobiliary tube, a drainage bag, a sealed cylinder, a heating mechanism, a filtering mechanism, and a reinfusion tube, wherein the drainage bag is disposed at an output end of the nasobiliary tube, the sealed cylinder is connected to an output end of the drainage bag, the heating mechanism and the filtering mechanism are disposed inside the sealed cylinder to heat and filter drained bile, and the reinfusion tube is connected to an output end of the sealed cylinder to reinfuse the heated and filtered bile into a patient;
the filtering mechanism comprises a first disc, a second disc, and a plurality of filter tubes, wherein the first disc and the second disc are spaced apart inside an input end of the sealed cylinder, the plurality of filter tubes are disposed in a circumferential array on the first disc and the second disc, and the filter tubes on the first disc and the second disc filter the bile entering the first disc and the second disc; and
the filter tubes comprise coarse filter tubes and fine filter tubes, wherein the coarse filter tubes and the fine filter tubes are both disposed in a circumferential array on the first disc and the second disc, the coarse filter tubes on the first disc are in communication with tube ports of the fine filter tubes on the second disc, and the fine filter tubes on the first disc are in communication with tube ports of the coarse filter tubes on the second disc.

2. The bile drainage and reinfusion device according to claim 1, wherein the heating mechanism comprises a heating layer and a plurality of electric heating rods, wherein the heating layer is cylindrically disposed inside the sealed cylinder, the plurality of electric heating rods are disposed in a circumferential array inside the heating layer, and the heating layer is filled with water for heating.

3. The bile drainage and reinfusion device according to claim 1, wherein the filtering mechanism further comprises a spring coil, and two side surfaces of the spring coil are fixedly disposed on the first disc and the second disc.

4. A bile drainage and reinfusion device, comprising: a nasobiliary tube, a drainage bag, a heating mechanism, a filtering mechanism, a reinfusion tube, and a circulation tube, wherein the drainage bag is disposed at an output end of the nasobiliary tube, the filtering mechanism is connected to an output end of the drainage bag, the heating mechanism is connected to an output end of the filtering mechanism and is configured to heat filtered bile, the reinfusion tube is disposed at an output end of the heating mechanism, the circulation tube is disposed at a tail end of the filtering mechanism, and an output end of the circulation tube is in communication with an input end of the filtering mechanism;
the heating mechanism comprises a plurality of electric heating rods, an outer cylinder, and an inner cylinder, wherein the outer cylinder is connected to the output end of the filtering mechanism, the inner cylinder is disposed inside the outer cylinder, and the plurality of electric heating rods are disposed in a circumferential array in a gap composed of the outer cylinder and the inner cylinder; and
the heating mechanism further comprises a support base, a rotating barrel, and a plurality of arc-shaped plates, wherein the support base is disposed inside the inner cylinder, the rotating barrel is movably sleeved on an outer periphery of the support base, and the plurality of arc-shaped plates are disposed in a circumferential array on an outer periphery of the rotating barrel.

5. The bile drainage and reinfusion device according to claim 4, wherein the filtering mechanism comprises a mounting cylinder, a mounting base, a plurality of ceramic filter tubes, and a sealing cap, wherein the mounting base is in communication with the output end of the drainage bag, the mounting cylinder is hermetically disposed on the mounting base, the plurality of ceramic filter tubes are disposed in a circumferential array on the mounting base, and the sealing cap is hermetically disposed on the plurality of ceramic filter tubes.

6. The bile drainage and reinfusion device according to claim 5, wherein the filtering mechanism further comprises a nut and a bearing, wherein the bearing is sleeved on an outer periphery of the sealing cap, the nut is disposed on an outer periphery of the bearing, an end of the mounting cylinder is provided with a thread adapted to the nut, and the nut drives the sealing cap via the thread to be fixed on the mounting cylinder.
